(19) United States
(12) Patent Application Publication
(10) Pub. No.: US 2007/0238990 A1
(43) Pub. Date: Oct. 11, 2007

(54) Control method as well as an imaging appliance

(76) Inventors: Gabriel Haras, Mucke (DE); Ute Feuerlein, Erlangen (DE)

Correspondence Address: HARNESS, DICKEY & PIERCE, P.L.C., P.O.BOX 8910, RESTON, VA, 20195, US

(21) Appl. No.: 11/650,434

(22) Filed: Jan. 8, 2007

(30) Foreign Application Priority Data
Jan. 9, 2006 (DE) 10 2006 001 090.6

(51) Int. Cl. A61B 5/05

(52) U.S. Cl. 600/431; 600/407

(57) ABSTRACT

A control method is specified for an imaging appliance, wherein a stored scan protocol in which operating parameters of the imaging appliance for image recording are stored, or a stored contrast agent protocol, in which parameters of contrast agent dosage are stored, is selected from a databank, contrast agent and scan protocols which are linked to the selected scan or contrast agent protocol are identified in the databank on the basis of a stored logic parameter which is associated with the respective protocols, and, if present, the or each protocol identified in this way is output as the contrast agent or scan protocol associated with the selected scan or contrast agent protocol. A correspondingly suitable imaging appliance is also specified.

### PRIORITY STATEMENT

The present application hereby claims priority under 35 U.S.C. §119 on German patent application number DE 10 2006 001 090.6 filed Jan. 9, 2006, the entire contents of which is hereby incorporated herein by reference.

### FIELD

Embodiments of the invention generally relate to a control method for an imaging appliance, in particular for creation of slice images of an object. Embodiments of the invention also generally relate to an imaging appliance, such as one which is particularly suitable for creation of slice images of an object and has a control unit, for example.

### BACKGROUND

An imaging appliance, in particular for creation of slice images, is used in particular to obtain information about the interior of the object being examined. By way of example, examination records and in particular slice images can be used to obtain valuable information about the position, the size and the structure of internal organs, of bone tissue or of other soft tissue in a patient. In particular, successive slice images can also be converted to a three-dimensional representation.

One imaging appliance which is particularly suitable for the recording of slice images is, for example, an X-ray computed tomography scanner, a magnetic resonance imaging scanner, a photon emission computed tomography scanner or a positron emission scanner. An imaging appliance such as this can be configured just as well on the basis of ultrasound.

The contrast in the object images produced by an imaging appliance such as this, for example of a patient, is caused by locally different excitation, absorption, reflection or emission characteristics of the materials being examined in comparison to the radiation, particle bombardment or sound waves used by the imaging appliance. In the case of an X-ray appliance, the various absorption or attenuation characteristics of different types of tissue are utilized to provide contrast. Since, for example, bone tissue and soft tissue differ greatly in terms of the stated characteristics, it is possible to analyze the structure of a bone in a patient on the basis of the image contrast associated with it.

Organs or vessels whose stated characteristics do not differ significantly with regard to the formation of contrast in the recorded images from the surrounding tissue cannot be examined by conventional devices/methods, because the resultant contrast is too low. For this reason, when examining an organ that is filled with blood, for example a heart, a liver or a vessel in the area of the extremities of the patient, a contrast agent is introduced into the patient's blood circulation before the start of the examination by the imaging appliance. The contrast agent results in the organs examined being imaged with sufficiently high contrast in comparison to the surrounding tissue.

When using a dose of contrast agent to create slice images, care must be taken to ensure the presence of the contrast agent at the same time. For this purpose, it is necessary to match the operating parameters of the imaging appliance and the propagation of the contrast agent that has been given in the patient to one another. For this purpose, a so-called scan protocol, in which operating parameters of the imaging appliance relating to image recording are stored, and a so-called contrast agent protocol, in which parameters of a contrast agent dosage are stored, are in each case selected such that they match one another for a desired examination area.

If, for example, the intention is to carry out angiography in the head area of the patient, then an appropriately suitable scan protocol is first of all selected for the imaging appliance. By way of example, performance values, the duration of the incident radiation, the feed rate, the radiation energy and the delay time between successive slice image records are stored as operating parameters in this scan protocol. In addition, the scan protocol can provide respectively appropriate operating parameters for selection for a predetermined patient constitution.

After selection of the suitable scan protocol, an appropriate contrast agent protocol is selected on the basis of existing empirical values, on the basis of whose parameters, for example, the dosage of the contrast agent required for recording purposes is provided by a contrast agent appliance. Parameters relating to the contrast agent dosage may, for example, be the concentration of the contrast agent, the flow of the contrast agent and the absolute amount of contrast agent to be supplied. A contrast agent protocol can also include a plurality of different phases of the contrast agent dosage. These phases may differ in the said parameters. In complex contrast agent protocols phases in which, for example, a saline solution is supplied can also be arranged between phases in which contrast agent is being given.

The contrast agent protocol is used to produce a predictable contrast agent profile in the patient for the recording of the images by the imaging appliance, so that the contrast agent concentration required for contrast imaging in the examination area exists during image recording. By way of example, a phase in which a saline solution is supplied is thus used to maintain the predictable contrast agent profile over time, without unnecessarily giving the patient too much contrast agent.

For examination purposes, the suitable scan protocol is then set or predetermined on the imaging appliance, and the appropriate contrast agent protocol for this is then set or predetermined on the contrast agent appliance. The examination is then carried out using the selected parameters.

Modern contrast agent appliances allow the storage of a limited number of basic contrast agent protocols. These basic contrast agent protocols can be individually matched to specific requirements, scan protocols and patients. In this case, the matching process is carried out on the basis of empirical values which, for example, may be stored in written form. The known procedure has the disadvantage that scan protocols and contrast agent protocols are not optimally combined. This can lead to the patient unavoidably being given an increased amount of contrast agent or being subjected to increased radiation or particle bombardment that is used by the imaging appliance.

US 2002/0071521 A1 describes a diagnostic system having a contrast agent injector, in which contrast conditions which are correlated with an examination method or examination region are output once this examination method or examination region has been entered.

### SUMMARY

In at least one embodiment of the invention, a control method is specified for an imaging appliance, by which errors in the selection of scan and contrast agent protocols can be avoided as far as possible. In at least one embodiment of the invention, an imaging appliance is specified, which can be operated using an advantageous control method such as this.

According to at least one embodiment of the invention, a control method is for an imaging appliance, wherein a stored scan protocol in which operating parameters of the imaging appliance for image recording are stored, or a stored contrast agent protocol, in which parameters of contrast agent dosage are stored, is selected from a databank, contrast agent and scan protocols which are linked to the selected scan or contrast agent protocol are identified in the databank on the basis of a stored logic parameter which is associated with the respective records, and, if present, the or each record identified in this way is output as the contrast agent or scan protocol associated with the selected scan or contrast agent protocol.

A first step of at least one embodiment of the invention is in this case based on the idea of combination of matching scan and contrast agent protocols in a databank. A further step of at least one embodiment of the invention is then based on the idea of associating the stored protocols with respective logic parameters, which are likewise stored, and of identification and outputting of suitable pairs of scan and contrast agent protocols on the basis of these logic parameters.

The logic parameters which are stored in the databank make it possible to link matching protocols with one another. The logic parameters may for this purpose include, in particular, empirical values, or else theoretically given relationships.

When a matching scan protocol is output on the basis of a predetermined contrast agent protocol, then this can be used automatically by the imaging appliance for the examination that is to be started. Conversely, if a matching contrast agent protocol is output on the basis of a scan protocol for examination of a desired area, then, for example, this can be transferred manually to a contrast agent appliance, or else can be transmitted automatically to it via an appropriate network. The last alternative in particular makes it possible for the imaging appliance to drive the contrast agent appliance appropriately at the start of the scanning process. The combination of the pairs of contrast agent and scan protocol, found can be selected directly as an examination protocol.

The identified scan or contrast agent protocols can, for example, be output as a print-out, as a file or directly as a data interchange between the connected appliances. In particular, they can also be output in the form of a display on a graphics display unit.

In one advantageous refinement, if there are a plurality of identified records, these are indicated as a selection, and are selected to be output by entering at least one of the identified records. A procedure such as this makes it possible to output a plurality of contrast agent and scan protocols in each case for one predetermined scan or contrast agent protocol, which, for example, are suitable for different physical patient constitutions. Thus, for example, a plurality of suitable contrast agent protocols can be output when a scan protocol is selected for recording of angiography in the head area of the patient, with these protocols differing in the contrast agent flow while it is being supplied, and to this extent relating to different patient cardiac minute volumes. This equally allows a procedure to be used on the basis of which a series of matching scan protocols are output following preselection of a contrast agent protocol which, for example, is suitable for angiography, with these scan protocols differing, for example, by their use for different patient body regions. This allows reliable user guidance, with incorrect associations being virtually precluded.

A new scan or contrast agent protocol is advantageously stored by way of an entry in the databank. A method step such as this makes it possible to continuously increase the number of protocols stored in the databank, and to this extent to broaden the knowledge base.

In a further refinement, a stored protocol and/or a stored logic parameter is modified by way of an entry in the databank. This not only makes it possible to modify protocols in the databank and to match them to new conditions, but also to modify their links to matching protocols.

In one advantageous variant, for a new entry or for modification of a scan or contrast agent protocol, the respectively possible operating parameters and/or the respectively possible parameters of a contrast agent dose are stated as a preselection, and the choice of the operating parameters or of the parameters of a contrast agent dose is made by way of an entry, and is stored in the databank, relating to the new or to the modified scan or contrast agent protocol.

This allows reliable user guidance by presenting the user with possible parameters for preselection. This allows the user to create scan or contrast agent protocols, from new, easily and directly on the imaging appliance, or to modify already existing scan or contrast agent protocols.

Furthermore, a logic parameter relating to a new or modified protocol is advantageously stored by way of an entry in the databank. This makes it possible for the user to decide whether to retain the existing logic parameter or modified protocols, or else to create new links on the basis of experience gained. The databank is matched to actual empirical values by the entry and storage of logic parameters.

The matching of the modified scan or contrast agent protocols to the user's own empirical values is made easier in that, after the modification of a scan or contrast agent protocol, the associated logic parameter or parameters is or are stated for selection, and is or are retained or modified by an entry in the databank. In this case, the user identifies the existing links to other protocols and can easily decide whether he wishes to change the existing links, to delete them or add new links to his modified protocol.

User information relating to the scan or contrast agent protocol is expediently stored. This makes it possible for a user to identify in particular his own protocols based on his empirical values, or else to find out which user has stored existing protocols.

In one further particularly advantageous refinement, the operator guidance is provided by menu assistance on a graphics display unit. In this case, the selection of protocols and of matching protocols which relate to selected protocols can be carried out easily, directly on the display unit. This is done either by way of a computer mouse, as has become normal practice in recent years, or else by the display unit being in the form of a touch screen. In this case, the entries which are required relating to the control method procedure can be activated by clicking or touching appropriately configured fields.

In one advantageous variant, a scan or contrast agent protocol is imported or exported by way of the data interchange between the internal databank and an external databank. This allows stored protocols to be used at a different point and, to this extent, allows a large knowledge pool to be used jointly. In particular, the last method step allows the contrast agent protocols which match a predetermined scan protocol to be passed on directly to a corresponding contrast agent appliance. There is no longer any need to transfer the proposed contrast agent protocol by hand.

According to at least one embodiment of the invention, an imaging appliance includes a control unit, wherein the control unit is designed to indicate for selection from a databank a stored scan protocol, in which operating parameters of the imaging appliance relating to image recording are stored, or a stored contrast agent protocol, in which parameters of contrast agent dosage are stored, to detect a selection and to identify linked contrast agent and/or scan protocols relating to the selected scan or contrast agent protocol in the databank, on the basis of a stored logic parameter which is associated with the respective protocols, and, if present, to output the or each protocol identified in this way as the contrast agent or scan protocol associated with the selected scan or contrast agent protocol.

### BRIEF DESCRIPTION OF THE DRAWINGS

Further advantageous refinements of the imaging appliance can be found below. In this case, advantages stated for at least one embodiment of a control method can be transferred in the same sense to advantageous refinements of at least one embodiment of the imaging appliance.

Example embodiments of the invention will be explained in more detail with reference to two tables and one drawing, in which:

Table 1 shows a graphics display for selection of an examination protocol,

Table 2 shows a graphics display for selection of a contrast agent protocol, and

FIG. 1 shows a perspective illustration of an X-ray computed tomography scanner, with an associated control unit, as an imaging appliance.

### DETAILED DESCRIPTION OF THE EXAMPLE EMBODIMENTS

It will be understood that if an element or layer is referred to as being “on”, “against”, “connected to”, or “coupled to” another element or layer, then it can be directly on, against, connected or coupled to the other element or layer, or intervening elements or layers may be present. In contrast, if an element is referred to as being “directly on”, “directly connected to”, or “directly coupled to” another element or layer, then there are no intervening elements or layers present. Like numbers refer to like elements throughout. As used herein, the term “and/or” includes any and all combinations of one or more of the associated listed items.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

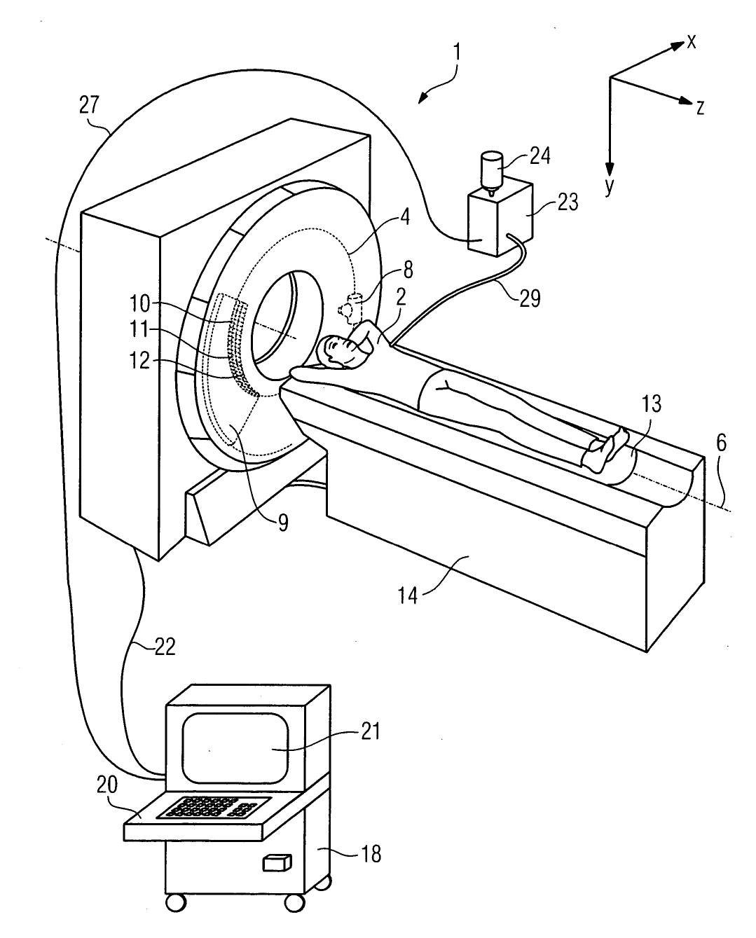

As an imaging appliance, FIG. 1 shows a computed tomography scanner 1 for examination of an object, in this case a patient 2. The computed tomography scanner 1 has a radiation source 8, which is arranged in a gantry 4 such that it can rotate about a rotation axis 6, for emission of X-ray radiation. The radiation source 8 is arranged opposite a curved detector 9, which has a plurality of detector elements arranged in rows to form detector rows 10, 11, 12.

The computed tomography scanner 1 also has a tabletop 13, which is mounted on a table 14 such that it can be moved along the rotation axis 6.

A control unit 18 is shown as a further component of the computed tomography scanner 1, and has a control console 20 and a graphics display unit 21. The control unit 18 is connected to the computed tomography scanner 1 via a control line 22.

The control unit 18 is designed to control the forward movement of the moving tabletop 13 and the rotation of the gantry 4 in order to record successive slice images. The gantry 4 and tabletop 13 together form a positioning unit, which make it possible to record slice images with the patient 2 in different physical positions. The scanning rate of the computed tomography scanner 1 can be adjusted via the pitch value, that is to say the ratio of the rotation rate of the gantry 4 and the forward movement speed of the tabletop 13. In this case, the rotation of the gantry 4 ensures the recording of a slice image at one longitudinal position of the patient 2 while, in contrast, the forward movement of the tabletop 13 is responsible for the sequence of slice images to be recorded.

In order to record a slice image, the X-ray radiation which has been emitted from the radiation source 8 and has passed through the patient 2 is recorded by the detector 9. In the case of the illustrated computed tomography scanner 1, the radiation source 8 produces a fan-shaped X-ray beam for this purpose. A characteristic attenuation image of the X-ray radiation is thus recorded in each position of the gantry 4. A slice image is reconstructed from the projections obtained in different positions of the gantry 4, on which tissue with different attenuation characteristics is represented by different gray-scale values.

These slice images are recorded by control of the gantry 4, of the tabletop 13 and of the radiation source 8 on the basis of scan protocols selected on the control unit 18. In this case, the scan protocols include performance values for the radiation source 8, forward-movement parameters, in particular such as a pitch value, as well as a scan delay value. In order to select the scan protocols, these are displayed on the display unit 21, sorted by way of example on the basis of examination areas. In this case, the display unit 21 has a touch screen, so that the displayed scan protocols are selected by manual touching.

A contrast agent dose is provided in order to examine organs filled with blood, for example a heart, a liver or a blood vessel. For this purpose, the control unit 18 is connected via a control line 27 to a contrast agent appliance 23, via which a contrast agent 24 is supplied in a controlled manner to the patient 2, by way of a flexible contrast agent tube 29, in accordance with a predetermined contrast agent protocol.

The control unit 18 offers menu-assisted operator guidance for selection of the contrast agent protocol, which includes the number and nature of different dosage phases as contrast agent dosage parameters, with these phases each differing in the nature of the agent supplied, its flow and the absolute amount. After the predetermined selection of the desired scan protocol, the control unit selects associated contrast agent protocols on the basis of logic parameters stored in a databank, and offers these to the user for selection on the display unit 21. The displayed contrast agent protocols are in this case intended for different patient constitutions. The user then selects the appropriate contrast agent protocol for the given constitution of the patient 2 by touching the touch screen at an appropriate point. The control unit 18 then transmits the selected contrast agent protocol to the contrast agent appliance 23, via the control line 27.

When the examination is started, the scan protocol and the contrast agent protocol are thus matched to one another. The slice images to be recorded therefore have good contrast.

The control unit 18 also makes it possible to enter new scan or contrast agent protocols with menu assistance, or to modify already existing protocols. Possible operating parameters for selection are predetermined for a new entry. The user can enter the selection by appropriately touching the touch screen. In order to modify existing protocols, both the stored parameters and the associated logic parameters are displayed as a selection. The user can vary any of the displayed parameters, can delete them or, if required, can add a parameter that does not exist. User information relating to the newly entered or modified protocols can also be stored, allowing subsequent identification. Overall, this results in a knowledge base which, inter alia, is also based on empirical values and in which scan and contrast agent protocols are linked appropriately to one another being created in the control unit 18.

TABLE 1

| Examination protocol | Scan protocol | | Contrast agent | | |
|---|---|---|---|---|---|
| | | | protocol | | |
| | Head CT angio | | CT angio | | |
| Head: | mAs | 160 | Phase 1: | | |
| Brain | kV | 100 | Conc.: | 300 | |
| Face | Scan delay | 18 s | Flow: | 3.5 | ml/s |
| CT angio | ... | | Amount: | 70 | ml |

| | |
|---|---|
| ... | ... |

| Thorax: | Phase 2: | | |
|---|---|---|---|
| Lungs | Conc.: | NaCL | |
| Heart | Flow: | 3.4 | ml/s |
| ... | Amount: | 20 | ml |

Abdomen:
Liver
CT angio

Table 1 shows an example of a selection menu as is displayed on the display unit 21 of the control unit 18 for selection of the scan and contrast agent protocol that is suitable for a planned examination. By way of example, the user uses a computer mouse to click on the “Examination protocol” field at the top on the left, or touches this field in a corresponding manner on a touch screen. He then decides to select a head angiography, for which purpose he makes the “Head, CT angio” selection in the “Examination protocol” field.

This selection results in the scan protocol that is required for this intended head angiography being displayed in the “Scan protocols” column. A power of 160 mAs and an energy of 100 kV (keV) are preselected for the radiation source. The selected scan protocol is associated with a scan delay of 18 seconds. Further operating parameters, such as the pitch value, can follow.

After selection of the desired examination, the control unit 18 also outputs the selected scan protocol for contrast agent protocols associated with a head angiography. These are shown in the “Contrast agent protocol” column. In this case, the contrast agent protocol is one which can be used generally for CT angiography. This includes two phases of contrast agent dosage. A concentration of 300 mg/ml, a flow of 3.5 ml/s and an absolute amount of 70 ml are indicated for the contrast agent in a phase 1. A phase 2 then takes place, in which a total amount of 20 ml of sodium chloride is supplied at a flow rate of 3.5 ml/s. The displayed contrast agent protocol may include further phases or contrast agent dosage parameters.

TABLE 2

| Examination protocol | Scan protocol | | Contrast agent |
|---|---|---|---|
| | | | protocol |
| | Head CT angio | | |
| Head: | mAs | 160 | Protocol 1 |
| Brain | kV | 100 | Protocol 2 |
| Face | Scan delay | 18 s | CT angio |
| CT angio | ... | | Protocol 4 |

Thorax:
Lungs
Heart
...
Abdomen:
Liver
CT angio
...

Table 2 shows a menu such as that which is output graphically on the display unit 21 of the control unit 18 for selection of a contrast agent protocol. In contrast to Table 1, the user has in this case selected the “Contrast agent protocol” field by selection by way of a computer mouse or by touching the touch screen at a suitable point. A series of protocols which are characterized, for example, by their names are then displayed to him in a column located underneath this. In the present case, the user selects the contrast agent protocol with the stored name “CT angio”.

After appropriate selection of the contrast agent protocol “CT angio”, all of the scan protocols which are linked via stored logic parameters with the selected contrast agent protocol appear in the “Scan protocol” column. By way of example, the scan protocol for recording of head angiography is shown here. The parameters correspond to those in Table 1.

The displayed selection in this case means that different scan protocols are possible with the selected contrast agent protocol. In particular on the basis of Table 2, the user has decided to select head angiography by selecting the head as the examination area in the “Examination protocol” column. It would also have been just as possible to select the scan protocol associated with this for carrying out the examination by selection of the “Thorax” examination area.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks™, cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A control method for an imaging appliance, comprising:
selecting from a databank, at least one of a stored scan protocol, in which operating parameters of the imaging appliance for image recording are stored, and a stored contrast agent protocol, in which parameters of contrast agent dosage are stored;
identifying in the databank, at least one of contrast agent and scan protocols linked to the selected scan and contrast agent protocol, the identifying being done on the basis of a stored logic parameter associated with the respective protocols; and
outputting at least one protocol, identified in this way, as the at least one of contrast agent and scan protocol associated with the at least one of selected scan and contrast agent protocol.

2. The control method as claimed in claim 1, wherein, if there are a plurality of identified protocols, these are indicated as a selection, and are selected to be output by entering at least one of the identified protocols.

3. The control method as claimed in claim 1, wherein at least one of a new scan and contrast agent protocol is stored by way of an entry in the databank.

4. The control method as claimed in claim 1, wherein at least one of a stored protocol and a stored logic parameter is modified by an entry in the databank.

5. The control method as claimed in claim 3, wherein, for at least one of a new entry and modification of at least one of a scan and contrast agent protocol, at least one of the respectively possible operating parameters and the respectively possible parameters of a contrast agent dose is stated as a preselection, and the choice of at least one of the operating parameters and the parameters of a contrast agent dose is made by way of an entry, and is stored in the databank, relating to the at least one of a new and modified at least one of a scan and contrast agent protocol.

6. The control method as claimed in claim 3, wherein a logic parameter relating to at least one of a new and modified protocol is stored by way of an entry in the databank.

7. The control method as claimed in claim 6, wherein, after the modification of at least one of a scan and contrast agent protocol, the at least one of associated logic parameter and parameters is stated for selection, and is at least one of retained and modified by an entry in the databank.

8. The control method as claimed in claim 1, wherein user information relating to the scan or contrast agent protocol is stored.

9. The control method as claimed in claim 1, wherein the operator guidance is provided by menu assistance on a graphics display unit.

10. The control method as claimed in claim 1, wherein at least one of a scan and contrast agent protocol is at least one of imported and exported by way of the data interchange between the internal databank and an external databank.

11. An imaging appliance, comprising:
a control unit designed
to indicate for selection from a databank at least one of a stored scan protocol, in which operating parameters of the imaging appliance relating to image recording are stored, and a stored contrast agent protocol, in which parameters of contrast agent dosage are stored,
to detect a selection and to identify linked at least one of contrast agent and scan protocols relating to the at least one of selected scan and contrast agent protocol in the databank, on the basis of a stored logic parameter which is associated with the respective records, and
if present, to output at least one of the records identified in this way as the at least one of contrast agent and scan protocol associated with the selected scan or contrast agent protocol.

12. The imaging appliance as claimed in claim 11, wherein the control unit is designed, if there are a plurality of identified records, to indicate these as a selection, to detect a selection, and to output this, corresponding to the selection of at least one of the identified protocols.

13. The imaging appliance as claimed in claim 11, wherein the control unit is designed to enter at least one of a new scan and contrast agent protocol and to store this in the databank.

14. The imaging appliance as claimed in claim 11, wherein the control unit is designed to modify at least one of a stored protocol and a stored logic parameter by way of an entry.

15. The imaging appliance as claimed in claim 13, wherein the control unit is designed to indicate at least one of the respectively possible operating parameters and the respectively possible parameters of a contrast agent dose as a preselection at least one of for a new entry and for modification of a scan or contrast agent protocol, to detect a selection of at least one of the operating parameters and the parameters of a contrast agent dose, and to store the selected at least one of operating parameter of a contrast agent dose in the databank relating to the at least one of new and the modified at least one of scan and contrast agent protocol.

16. The imaging appliance as claimed in claim 13, wherein the control unit is designed to enter a logic parameter relating to at least one of a new and modified protocol and to store the entered logic parameter in the databank.

17. The imaging appliance as claimed in claim 16, wherein the control unit is designed to indicate the at least one associated logic parameter for selection after the modification of at least one of a scan and contrast agent protocol, to detect a selection, and at least one of to retain and to modify the at least one of selected logic parameter in the databank.

18. The imaging appliance as claimed in claim 11, wherein the control unit is designed to store user information associated with at least one of the scan and contrast agent protocol.

19. The imaging appliance as claimed in claim 11, wherein a graphics display unit is provided, and the control unit is designed for menu-assisted user guidance on the graphics display unit.

20. The imaging appliance as claimed in claim 11, wherein the control unit is designed to at least one of import and export at least one of a scan and contrast agent protocol by interchanging data between the internal databank and an external databank.

21. The control method as claimed in claim 2, wherein at least one of a new scan and contrast agent protocol is stored by way of an entry in the databank.

22. The control method as claimed in claim 4, wherein, for at least one of a new entry and modification of at least one of a scan and contrast agent protocol, at least one of the respectively possible operating parameters and the respectively possible parameters of a contrast agent dose is stated as a preselection, and the choice of at least one of the operating parameters and the parameters of a contrast agent dose is made by way of an entry, and is stored in the databank, relating to the at least one of a new and modified at least one of a scan and contrast agent protocol.

23. A computer readable medium including program segments for, when executed on a computer device, causing the computer device to implement the method of claim 1.

24. The imaging appliance as claimed in claim 12, wherein the control unit is designed to enter at least one of a new scan and contrast agent protocol and to store this in the databank.
